(19) United States
(12) Patent Application Publication
(10) Pub. No.: US 2008/0262536 A1
(43) Pub. Date: Oct. 23, 2008

(54) APPARATUS FOR TREATING SOFT TISSUE INJURIES DURING PHYSICAL ACTIVITY

(75) Inventor: Eilaz Babaev, Minnetonka, MN (US)

Correspondence Address: Bacoustics, LLC, 5929 BAKER ROAD, SUITE 470, MINNETONKA, MN, 55345, US

(73) Assignee: AB Ortho, LLC, Minnetonka, MN (US)

(21) Appl. No.: 11/737,073

(22) Filed: Apr. 18, 2007

(51) Int. Cl. A61B 17/00

(52) U.S. Cl. 606/204

(57) ABSTRACT

An apparatus is provided for treating injured muscles during physical activity. Using the present involves pressing a plurality of support members against the skin proximate to a muscle for a sustained period of time. The support members pressed against have an arc spanning a base along at least one axis. The support members are secured to the therapeutic surface of a therapeutic body as to create the apparatus of the present invention. The base of support members is secured to the therapeutic surface such that the apexes of the support members' arcs extend away from the therapeutic surface.

### BACKGROUND OF THE INVENTION

1. Field of the Invention

The present invention relates to orthopedic devices and treatment methods. More particularly, the present invention relates to orthopedic devices and methods utilizing a plurality of discrete pressure points.

2. Description of the Related Art

Strained, fatigued, or otherwise injured muscles are generally treated by the use of non-steroidal anti-inflammatory drugs (NSAIDs) either prior to, after, or during physical activities.

External support devices, such as bandages, slings, or braces, are also often used to treat injured muscles prior to, after, or during physical activities. The aforementioned devices typically elicit a therapeutic benefit on injured muscles by providing support, inhibiting the massive movement of extensor and flexor muscles, absorbing shock, and may enable the warming or cooling of injured muscles.

Chinese acupuncture, well known and recognized worldwide, but not full appreciated, can also be used for treating injured muscles. During acupuncture therapy, needles are inserted into the body at defined bioactive points. The needles usually remain in the body for a defined period of time. The administration of acupuncture therapy requires a skilled, and in some localities a certified, practitioner.

Acupressure is a derivation of acupuncture therapy. During acupressure therapy, a skilled practitioner applies mechanical pressure to specific bioactive points, while being careful to not apply a harmful amount of pressure.

### SUMMARY OF THE INVENTION

The present invention is directed towards an apparatus for supporting muscles during physical activity. Capable of self-administration by the user, the present invention avoids the need of skilled and/or certified practitioners. Providing therapeutic pressure simultaneously along multiple points and/or in different directions, such as, but not limited to, radial, longitudinal, and/or any combination thereof, the present invention may elicit a superior therapeutic effect than conventional tension bandages. In addition to the previously enumerated advantages over the short comings of the existing devices and methods, the present invention may also provide additional advantages and improvements that will be recognizes by people of ordinary skill in the art upon review of the present disclosure.

The present invention comprises a therapeutic body, a therapeutic surface on side of the body, and a plurality of support members secured to the therapeutic surface. Each of the support members comprises a base secured to the therapeutic surface and arced cross section on at least one axis transecting the support member, with the apex of the arc extending away from the therapeutic surface.

During physical activity, the present invention is placed in contact with the body proximate to and/or over an injured muscle, with the therapeutic surface facing the body. A pressure is then applied to the present invention. The applied pressure is distributed amongst the support members contacting the body. Translating the pressure applied to the support member into a compressive stress, the arched cross-sections of the support members provide a supportive pressure to muscle.

The support members may elicit a therapeutic effect on a muscle proximate to and/or over which the present invention is pressed through a variety of mechanism of action. Providing supportive pressure, the support members may bring about pain relief in a manner similar to that of acupressure therapy. Supporting the muscle, the support members may absorb shock. Distributing the supportive pressure across the support members may cause the skin over the muscle to stretch as to open pores in the skin surface. The opening of pores in the skin surface may increase absorption of oxygen through the skin. Distributing supportive pressure across the support members may cause stretching of the muscle beneath the present the invention. Other therapeutic benefits and/of mechanisms of actions, in addition to those listed, may be elicited by the support members of the present invention.

### BRIEF DESCRIPTION OF DRAWINGS

FIG. 1 depicts three-dimensional views of two embodiments of the apparatus of the present invention and a cross sectional view common to both of the depicted embodiments.

FIG. 2 depicts a three-dimensional view an embodiment of the apparatus of the present invention secured to a strap.

FIG. 3 depicts a cross-sectional view of an embodiment of the apparatus of the present invention secured to a bladder.

### DETAILED DESCRIPTION

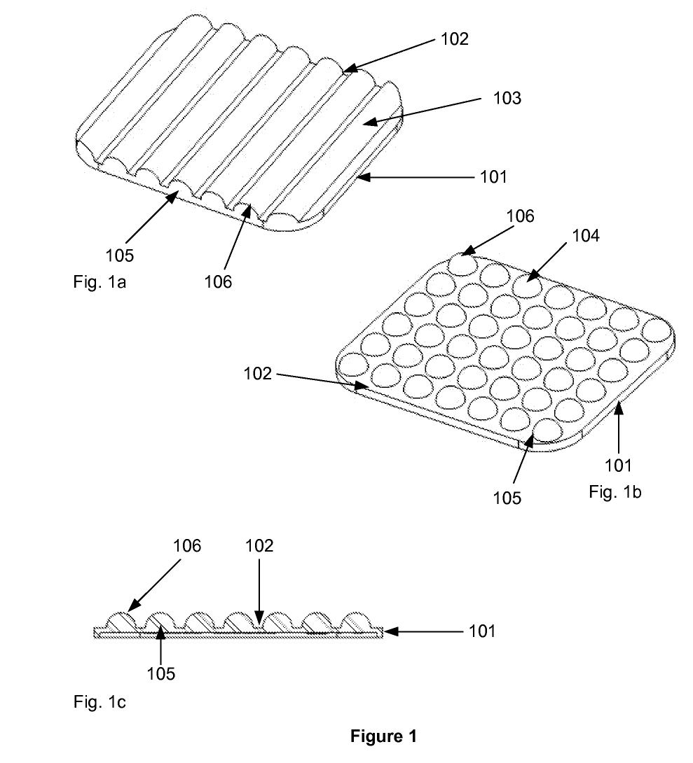

FIG. 1 depicts three dimensional views (FIG. 1a and FIG. 1b) of two embodiments of the muscle support apparatus of the present invention and a cross-sectional view (FIG. 1c) common to both of the depicted embodiments. The present invention comprises a therapeutic body 101, a therapeutic surface 102 on side of the therapeutic body 101, and a plurality of support members 103 and 104. Each of the support members 103 and 104 comprises a base 105 secured to the therapeutic surface, and an arc 106 spanning the base along at least one axis. As depicted in FIG. 1a, arc 106 may be extruded along a second axis forming vaults 103. The arc 106 may also be revolved along on axis forming domes 104, as depicted in FIG. 1b. Other support member configurations are possible and in accordance with the present invention, so long as there is an arc spanning at least one axis of the base.

Support members may be made from plastic and/or a polymer. The support members may also be made from a variety of materials. Precious metals such as, but not limited to, gold, silver, and/or platinum, may be used in constructing the support members.

Therapeutic body 101 may be made from a compliant material or rigid material. If a rigid material is used, it may be desirable to segment therapeutic body 101 as to enable therapeutic body 101 to conform to the wearer's body.

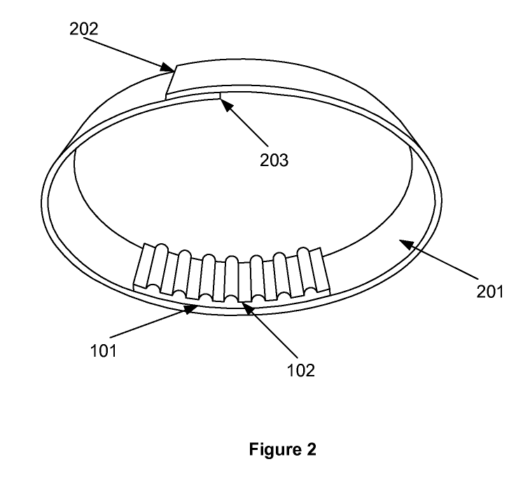

During and/or throughout physical activity, such as, but not limited to, exercise, playing sports, and/or manual labor, the present invention is pressed against the body, with therapeutic surface 102 facing the body. Preferably, the presenting invention is pressed proximate to and/or over an injured muscle. The present invention may be pressed against the body and/or secured in place with the use of a strap 201, depicted in FIG. 2. Strap 201 may be fastened around a portion of the body such as, but not limited to, a limb, a shoulder, trunk, and/or head by tying ends 202 and 203 together. Alternatively, Velcro attached to end 202 may be used to secure strap 201 to a portion of the body. Strap 201 may also be secured by the use of snaps, a buckle, buttons, and/or numerous other methods readily recognizable to a person of ordinary skill in the art. The present invention may also be secured in place and/or pressed against the body with the use of a sling, brace, bandage, tape, and/or numerous other devices readily recognizable to a person of ordinary skill in the art.

Regardless of the device used to secure and/or press the present invention against a portion of the body, it is preferable the present invention be secured to the device. Ideally, therapeutic body 101 is removably secured to strap 201 or a similar device. Therapeutic body 101 may be removably secured to strap 202 or a similar device by including an eye or plurality of eyes on therapeutic 101 through which strap 202 or a similar device is threaded. Therapeutic body 101 may also be removably secured to strap 202 or a similar device by attaching a piece of Velcro on therapeutic body 101 opposite therapeutic surface 102 and a mating piece of Velcro on strap 201 or a similar device. As an alternative to Velcro, temporary adhesives, snaps, and/or numerous other methods readily recognizable to person or ordinary skill in the art may be used to removably secure therapeutic body 101 to strap 201 or similar devices.

Vaulted support members 103 may be desirable when the wearer is experiencing pain localized to particular muscle or group of muscles during physical activity. If a therapeutic body 101 with a therapeutic surface 102 measuring 5 cm by 6 cm is used, seven vaults with a length of 5 cm, a width of 6 mm, and a height of 3 mm may be positioned uniformly over therapeutic surface 102, as depicted in FIG. 1a. Other vault dimensions and density are equally possible. Furthermore, therapeutic surface 102 may be larger or smaller than the size previously enumerated. Ideally, the present invention with vaulted support member 103 is positioned against the body proximate to and/or over the injured muscle. The present invention should, but need not, be oriented such that vaulted support members 103 run with the thread of the muscle or muscles.

Domed support members 104 may be desirable when the wearer is experiencing as shooting during physically activity. If a therapeutic body 101 with a therapeutic surface 102 measuring 5 cm by 6 cm is used, 42 domes with a base diameter of 6 mm and a height of 3 mm may be positioned uniformly over therapeutic surface 102, as depicted in FIG. 1b. Other dome dimensions and density are equally possible. Furthermore, therapeutic surface 102 may be larger or smaller than the size previously enumerated. Ideally, the present invention with domed support members 104 is position against the body positioned against the body proximate to and/or over the injured muscle from which the shooting pain originates.

Regardless of the configuration, size, or density, the support members may be secured to the therapeutic surface 102 in a variety patterns. Possible patterns of the support members on the therapeutic surface 102 include, but are not limited to, linear, sinusoidal, triangular, rectangular, circular, spiral, and/or any combination thereof. It some situations, it may be preferable to select a support member pattern that approximates and/or resembles the configuration, shape, and/or thread of the injured muscle. Therapeutic body 101 and/or therapeutic surface 102 may be configured to match the support pattern utilized.

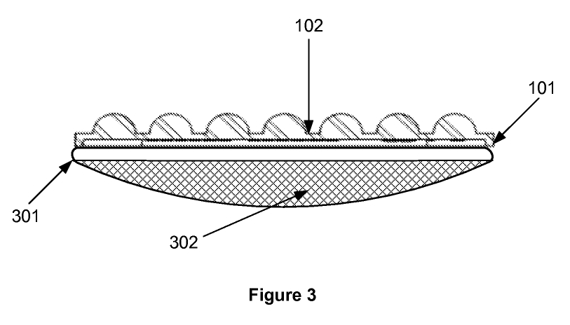

As depicted in FIG. 3, a bladder 301 may be secured to therapeutic body 101 opposite therapeutic surface 102. The bladder may be filled with a substance 302 hotter or colder than the body such as, but not limited, gas, solid, fluid and/or gel. The substance 302 within the bladder may contain chemicals that react exothermically or endothermically. Such chemicals are readily recognizable to a people of ordinary skill in the art. When bladder 301 is filled with a substance 302 hotter than the body and/or chemicals reacting exothermically, heat from bladder 301 will be transferred to muscles in contact with and/or proximate to the present invention. Conversely, when bladder 301 is filled with a substance 302 colder than the body and/or chemicals reacting endothermically, heat will be transferred from muscles in contact with and/or proximate to the present invention to bladder 301. Transferring heat from bladder 301 to injured muscles may elicit several therapeutic benefits such as, but not limited to, increasing blood flow to the muscles, and/or relaxing the muscles. Transferring heat from injured muscles to bladder 301 may elicit several therapeutic benefits such as, but not limited to, decreasing swelling, decreasing inflammation, and/or dulling pain.

Arc as used herein refers to a segment of a differentiable curve.

It should be appreciated that elements described with singular articles such as “a”, “an”, and “the” or otherwise described singularly may be used in plurality. It should also be appreciated that elements described in plurality may be used singularly.

Although specific embodiments of apparatuses and methods have been illustrated and described herein, it will be appreciated by people of ordinary skill in the art any arrangement, combination, and/or sequence that is calculated to achieve the same purpose may be substituted for the specific embodiments shown. It is to be understood that above description is intended to be illustrative and not restrictive. Combinations of the above embodiments and other embodiments as wells as combinations and sequences of the above methods and other methods of use will be apparent to individuals possessing skill in the art upon review of the present disclosure. The scope of the present invention should be determined with reference to the appended claims, along with the full scope of equivalents to which such claims are entitled.

I claim:

1. An apparatus for supporting muscles during physical activity comprising:
a. therapeutic body;
b. a therapeutic surface on one side of the therapeutic body; and
c. a plurality of support members each comprising:
i. a base secured to the therapeutic surface; and
ii. an arced cross section on at least one axis transecting the support member;
iii. wherein the apex of the arced cross section extends away the therapeutic surface;

2. The apparatus of claim 1, wherein at least one of the support members is domed.

3. The apparatus of claim 1, wherein at least one of the support members is vaulted.

4. The apparatus of claim 1, wherein the therapeutic body is compliant.

5. The apparatus of claim 1, further comprising a bladder secured to the therapeutic body opposite the therapeutic surface.

6. The apparatus of claim 5, further comprising a substance within the bladder.

7. The apparatus of claim 6, wherein thermal energy is transferred between a substance in the bladder and a muscle.

8. The apparatus of claim 1, further comprising a strap secured to the therapeutic body.
